(19) United States
(12) Patent Application Publication
(10) Pub. No.: US 2026/0151264 A1
(43) Pub. Date: Jun. 4, 2026

(54) OPHTHALMIC SURGICAL INSTRUMENT FOR INCISING FIBROUS TISSUE IN EYE

(71) Applicant: MANI, INC., Utsunomiya-shi (JP)

(72) Inventors: Toru SUGAWARA, Utsunomiya-shi (JP); Kenta TAKIZAWA, Utsunomiya-shi (JP)

(73) Assignee: MANI, INC., Utsunomiya-shi (JP)

(21) Appl. No.: 19/398,330

(22) Filed: Nov. 24, 2025

(30) Foreign Application Priority Data
Nov. 29, 2024 (JP) 2024-208131

(51) Int. Cl. A61F 9/007

(52) U.S. Cl. CPC A61F 9/007

(57) ABSTRACT

An ophthalmic surgical instrument for incising fibrous tissue in the eye, the ophthalmic surgical instrument including: a shaft having a longitudinal axis; and an incising portion projecting in a direction away from a longitudinal direction of the shaft, in which the shaft includes: a shaft front surface located on a side of a projection direction of the incising portion; and a shaft rear surface located on the opposite side from the projection direction, in which the incising portion includes: a tip having a minimum thickness to which a thickness of the incising portion in a longitudinal direction of the shaft is minimized toward the projection direction; a top surface being a connecting surface between the tip and the shaft front surface; and a bottom surface being a connecting surface between the tip and the shaft rear surface.

### BACKGROUND OF THE INVENTION

### Field of the Invention

The present invention relates to an ophthalmic surgical instrument, particularly an ophthalmic surgical instrument for incising fibrous tissue in the eye.

### Description of the Related Art

The trabecular meshwork is located between the anterior chamber of the eye and Schlemm's canal and functions as a filter through which aqueous humor passes before flowing into Schlemm's canal.

It is known that when dysfunction of the trabecular meshwork occurs, the outflow of aqueous humor is obstructed, resulting in increased intraocular pressure, which causes glaucoma. One cause of such dysfunction is an age-related loss of elasticity of the trabecular meshwork.

Surgical removal of the trabecular meshwork is known as one of the treatments for obstruction of aqueous humor outflow caused by dysfunction of the trabecular meshwork.

WO 2019/083903 discloses a device for incising a trabecular meshwork, the device comprising: a platform for elevating a portion of the trabecular meshwork away from an outer wall of a Schlemm's canal, the platform comprising a tip at a distal side of the platform and a top surface extending from the distal side to a proximal side of the platform, opposite the distal side of the platform; first and second sides extending from the top surface, wherein the first and second sides are parallel to each other; a bottom surface, opposite the top surface and defined by: a first protrusion below the first side; and a second protrusion below the second side, wherein the first and second protrusions are separated by a gap; and first and second lateral elements for creating first and second incisions through the trabecular meshwork, the first and second lateral elements extending from the proximal side of the platform.

### SUMMARY OF THE INVENTION

### Problems to be Solved by the Invention

However, the device disclosed in WO 2019/083903 involves a risk of damaging non-resectable tissues (such as the sclera and Schlemm's canal) as the insertion angle relative to the device insertion port in the eye increases.

### Means of Solving the Problems

The inventors have developed an ophthalmic surgical instrument in which the thickness of the shaft is locally reduced and an incising portion for incising fibrous tissue is shifted with respect to the shaft axis.

Such an ophthalmic surgical instrument can reduce the risk of damaging non-resectable tissues even when used at a larger insertion angle than conventional ophthalmic surgical instruments, whereby the present invention has been accomplished.
[1] It is an object of the present invention to provide an ophthalmic surgical instrument for incising fibrous tissue in the eye, the ophthalmic surgical instrument including:
a shaft having a longitudinal axis; and
an incising portion projecting in a direction away from a longitudinal direction of the shaft,
in which the shaft includes:
   a shaft front surface located on a side of a projection direction of the incising portion; and
   a shaft rear surface located on the opposite side from the projection direction,
in which the incising portion includes:
   a tip having a minimum thickness to which a thickness of the incising portion in a longitudinal direction of the shaft gradually decreases toward the projection direction;
   a top surface being a connecting surface between the tip and the shaft front surface; and
   a bottom surface being a connecting surface between the tip and the shaft rear surface, and
in which the shaft having, in a side view, a standard cross-section being perpendicular to a horizontal tangent that first becomes parallel to a horizontal axis extending in the longitudinal direction of the shaft among tangents to a surface or a point transitioning from the top surface to the shaft front surface and passing through a point of intersection between the horizontal tangent and the top surface, and
a center point of the standard cross-section is positioned rearward, toward the shaft rear surface, relative to a first axis passing through most number of center points in a front view among center points of a plurality of reference cross-sections of the shaft parallel to the standard cross-section.

The ophthalmic surgical instrument according to the present invention can reduce the risk of damaging non-resectable tissues even when used at a larger insertion angle than conventional ophthalmic surgical instruments.
[2] In the ophthalmic surgical instrument according to [1], the shaft may include a region having a constant thickness on the proximal side,
in which the thickness of the shaft may be smallest at the point of intersection and may increase proximally from the point of intersection up to the region of constant thickness.
[3] In the ophthalmic surgical instrument according to [1] or [2], the incising portion may further include:
a first cutting surface provided between the tip and the bottom surface; and
a second cutting surface provided between the tip and the top surface,
in which, in a side view, a second angle defined between the first axis and a second axis bisecting a first angle defined between the first cutting surface and the second cutting surface may be between 60 and 100 degrees on the side of the top surface side.
[4] It is another object of the present invention to provide an ophthalmic surgical instrument for incising fibrous tissue in the eye, the ophthalmic surgical instrument including:
a shaft having a longitudinal axis; and
an incising portion projecting in a direction away from a longitudinal direction of the shaft,
in which the shaft includes:
   a shaft front surface located on a side of a projection direction of the incising portion;
   a shaft rear surface located on the opposite side from the projection direction; and
   shaft side surfaces, each shaft side surface being a connecting surface between the shaft front surface and the shaft rear surface,
in which the incising portion includes:
   a tip having a minimum thickness to which a thickness of the incising portion in a longitudinal direction of the shaft gradually decreases toward the projection direction;
   a top surface being a connecting surface between the tip and the shaft front surface; and
   a bottom surface being a connecting surface between the tip and the shaft rear surface;
   incising side surfaces, each incising side surface being a connecting surface between the top surface and the bottom surface,
in which the shaft having, in a side view, a standard cross-section being perpendicular to a horizontal tangent that first becomes parallel to a horizontal axis extending in the longitudinal direction of the shaft among tangents to a surface or a point transitioning from the top surface to the shaft front surface and passing through a point of intersection between the horizontal tangent and the top surface, and
a center point of the standard cross-section is positioned rearward, toward the shaft rear surface, relative to a first axis passing through most number of center points in a front view among center points of a plurality of reference cross-sections of the shaft parallel to the standard cross-section, and
in which the top surface, bottom surface, shaft front surface and/or shaft rear surface include a surface roughness different from that of the incising side surfaces and/or shaft side surfaces.

The ophthalmic surgical instrument according to the present invention enables easier distinction between the side surfaces and the other surfaces than conventional ophthalmic surgical instruments, thereby facilitating identification of the orientation of the instrument during surgery.

### BRIEF DESCRIPTION OF THE DRAWINGS

FIG. 1 is a perspective view showing the front, right side, and top view of an ophthalmic surgical instrument 1 provided with a handle 2;

FIG. 2 is a perspective view showing the front view, right side, and top view of the ophthalmic surgical instrument 1;

FIG. 3 is an enlarged right side view of a distal region 10A of a shaft 10;

FIG. 4A shows the front, right side, and top views illustrating six cross-sections (standard cross-section CS_{S} and reference cross-sections CS_{R} (CS_{R1} to CS_{R5})) of the ophthalmic surgical instrument 1;

FIG. 4B shows a set of front views of each cross-section shown in FIG. 4A;

FIG. 5 is a schematic enlarged right side view of an incising portion 20;

FIG. 6 is a schematic view showing incision ranges of the ophthalmic surgical instrument 1 according to the present embodiment and a conventional ophthalmic surgical instrument 100;

FIG. 7A is a photograph showing a distal end of shaft 1, whose entire surface is processed as a glare surface (mirror-finished surface);

FIG. 7B is a photograph showing the distal end of shaft 1 where the top surface 22, bottom surface 23, shaft front surface 11, and shaft rear surface 12 are processed as non-glare surfaces (matte surface), and incising side surfaces 24 and shaft side surfaces 13 are processed as glare surfaces (mirror-finished surface);

FIG. 7C is a photograph showing the distal end of shaft 1, where the top surface 22, bottom surface 23, shaft front surface 11, and shaft rear surface 12 are processed as a glare surface (mirror-finished surface), and incising side surfaces 24 and shaft side surfaces 13 are processed as a non-glare surface (matte surface); and

FIG. 7D is a photograph showing the distal end of shaft 1, whose entire surface is processed as a non-glare surface (matte surface).

### DESCRIPTION OF THE EMBODIMENTS

### Definition

For convenience, certain terms employed in the context of the present disclosure are collected here. Unless defined otherwise, all technical and scientific terms used herein have the same meaning as commonly understood by one of the ordinary skilled in the art to which this invention belongs. The singular forms “a”, “an”, and “the” are used herein to include plural referents unless the context clearly dictates otherwise.

Notwithstanding that the numerical ranges and parameters setting forth the broad scope of the invention are approximations, the numerical values set forth in the specific examples are described as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art.

The term “side view” as used herein refers to a viewpoint in which, compared with other viewpoints, the longitudinal shape of an ophthalmic surgical instrument 1 described below is most clearly shown, and in which the projection direction of an incising portion 20 described below is most clearly shown as being substantially perpendicular to the longitudinal direction of the ophthalmic surgical instrument 1. The term “side view” includes a “right side view” and a “left side view.”

The term “front view” as used herein refers to a viewpoint in which, compared with other viewpoints, a surface of a bottom surface 23 described below is most clearly shown, and a surface of a top surface 22 described below is not shown.

The term “distal” as used herein refers to a direction along the longitudinal direction of the ophthalmic surgical instrument 1 in which the incising portion 20 is provided, and the term “proximal” as used herein refers to a direction opposite to “distal”.

The term “width” as used herein refers to a direction between side surfaces (a shaft side surface 13 and an incising side surface 24 described below) of a portion to which the term is applied.

Hereinafter, embodiments of the present invention are illustrated in detail. The following embodiments are illustrative only and do not limit the scope of the present invention. In order to avoid redundancy, explanation for similar contents is not repeated.

### Ophthalmic Surgical Instrument 1

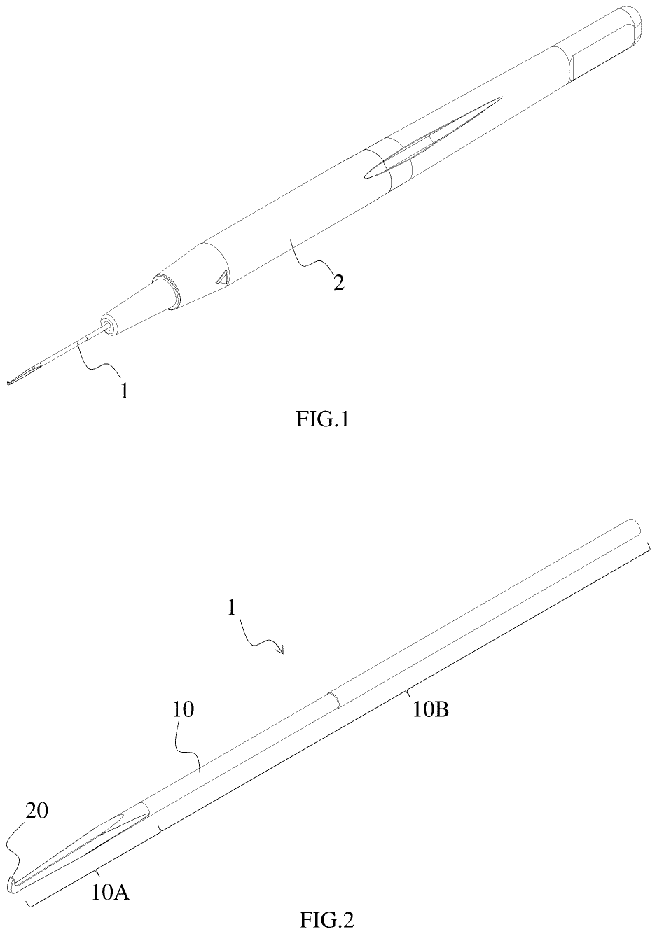

The ophthalmic surgical instrument 1 according to this embodiment is a surgical instrument for incising fibrous tissue within the eye. FIG. 1 is a perspective view showing the front, right side, and top view of the ophthalmic surgical instrument 1 provided with a handle 2. Since the ophthalmic surgical instrument 1 is sized to be insertable into the eye, it is used by being fitted into the handle 2 that is larger in size than the ophthalmic surgical instrument 1 for ease of manipulation by the surgeon. The longitudinal length of the ophthalmic surgical instrument 1 is approximately 120 mm, but is not limited thereto. The width and thickness of the ophthalmic surgical instrument 1 vary depending on the portion of the ophthalmic surgical instrument 1, but are approximately 0.1 mm to 7 mm; however, these ranges are not limiting.

The material of the ophthalmic surgical instrument 1 may be any material widely used for surgical instruments, such as stainless steel. The ophthalmic surgical instrument 1 can be manufactured by processing (e.g., cutting) a stainless-steel wire that has been rolled by pressing.

FIG. 2 is a perspective view showing the front view, right side, and top view of the ophthalmic surgical instrument 1. The ophthalmic surgical instrument 1 includes a shaft 10 having a longitudinal axis, and the incising portion 20 projecting in a direction away from the longitudinal direction of the shaft 10.

### Shaft 10

The shaft 10 according to this embodiment is divided into a distal region 10A and a proximal region 10B. The incising portion 20 is provided at the distal end of the distal region 10A. The proximal region 10B is longer than the distal region 10A, has a cylindrical shape, and constitutes a region of the shaft 10 that is in contact with the handle 2. The proximal region 10B preferably has a constant diameter, but may alternatively include a plurality of regions having different diameters. When the proximal region 10B includes a plurality of regions having different diameters, the central axes of the respective regions are preferably aligned on the same axis.

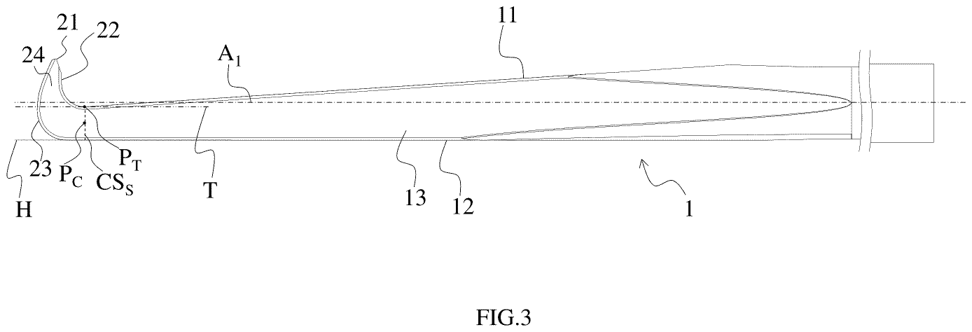

FIG. 3 is an enlarged right side view of the distal region 10A of the shaft 10. The distal region 10A of the shaft 10 according to this embodiment changes in shape from a cylindrical shape to a prismatic shape from the proximal side toward the distal side.

The distal region 10A of the shaft 10 includes a shaft front surface 11 located on the side of the projection direction of the incising portion 20, a shaft rear surface 12 located on the opposite side from the projection direction, and the shaft side surfaces 13, each shaft side surface 13 being a connecting surface between the shaft front surface 11 and the shaft rear surface 12. The thickness of the distal region 10A of the shaft 10 according to this embodiment (i.e., the length between the shaft front surface 11 and the shaft rear surface 12) increases toward the proximal side. The shaft front surface 11, shaft rear surface 12, and shaft side surfaces 13 according to this embodiment are substantially flat.

In one embodiment, the shaft 10 has a structure without a through hole. In another embodiment, the distal region 10A of the shaft 10 does not include a through hole. In still another embodiment, the shaft front surface 11 of the shaft 10 does not include a through hole.

### Cleaving Portion 20

The incising portion 20 according to this embodiment includes: a tip 21 having a minimum thickness to which the thickness of the incising portion 20 in the longitudinal direction of the shaft 10 gradually decreases toward the projection direction; the top surface 22 being a connecting surface between the tip 21 and the shaft front surface 11; and the bottom surface 23 being a connecting surface between the tip 21 and the shaft rear surface 12; and the incising side surfaces 24, each incising side surface 24 being a connecting surface between the top surface 22 and the bottom surface 23.

In one embodiment, the top surface 22 is curved so as to be concave toward the distal side when viewed in a top view. In one embodiment, the bottom surface 23 is curved so as to be convex toward the distal side when viewed in a top view. In one embodiment, the width of the top surface 22 is constant. In one embodiment, the width of the bottom surface 23 is constant. In one embodiment, the tip 21 is positioned on the proximal side relative to the distal end of the ophthalmic surgical instrument 1 when viewed in a side view.

In one embodiment, the width of the tip 21 is the same length as the width of the top surface 22 and/or the bottom surface 23. In another embodiment, the width of the tip 21 may be 95.0% to 99.9% (e.g., 95.0%, 96.0%, 97.0%, 98.0%, 99.0%, 99.1%, 99.2%, 99.3%, 99.4%, 99.5%, 99.6%, 99.7%, 99.8%, and 99.9%, or a range between any two values selected from these) of the width of the top surface 22 and/or the bottom surface 23.

### Center Point P_{C} of Standard Cross-Section CS_{S}

In the ophthalmic surgical instrument 1 according to this embodiment, when viewed in a side view, the shaft having a standard cross-section CS_{S} being perpendicular to a horizontal tangent T that first becomes parallel to a horizontal axis H extending in the longitudinal direction of the shaft 10 among tangents to a surface or a point transitioning from the top surface 22 to the shaft front surface 11 and passing through a point of intersection P_{T} between the horizontal tangent T and the top surface 22. The center point P_{C} of the standard cross-section CS_{S} is positioned rearward, toward the shaft rear surface 13, relative to the first axis A₁ passing through most number of center points P in a front view among center points P of a plurality of reference cross-sections CS_{R} of the shaft parallel to the standard cross-section CS_{S}. The horizontal axis H according to this embodiment is an axis parallel to the shaft rear surface 12 when viewed in a side view; however, it is preferably set to correspond to the insertion direction of the ophthalmic surgical instrument 1 into the eye or to a portion parallel to the insertion direction.

In one embodiment, the thickness of the shaft 10 is thinnest at the point of intersection P_{T}. In one embodiment, the thickness of the shaft 10 increases in the proximal direction from the point of intersection P_{T}. In one embodiment, the shaft 10 has a region having a constant thickness and/or width on the proximal side. In one embodiment, the shaft 10 has a region having a constant thickness and/or width on the proximal side, the thickness of the shaft 10 is thinnest at the point of intersection P_{T}, and the thickness of the shaft 10 increases in the proximal direction from the point of intersection P_{T} up to the region where the thickness is constant. In one embodiment, the width of the shaft 10 is constant. In one embodiment, the point of intersection P_{T} is located at a boundary between the top surface 22 and the shaft front surface 11.

### First Axis A₁

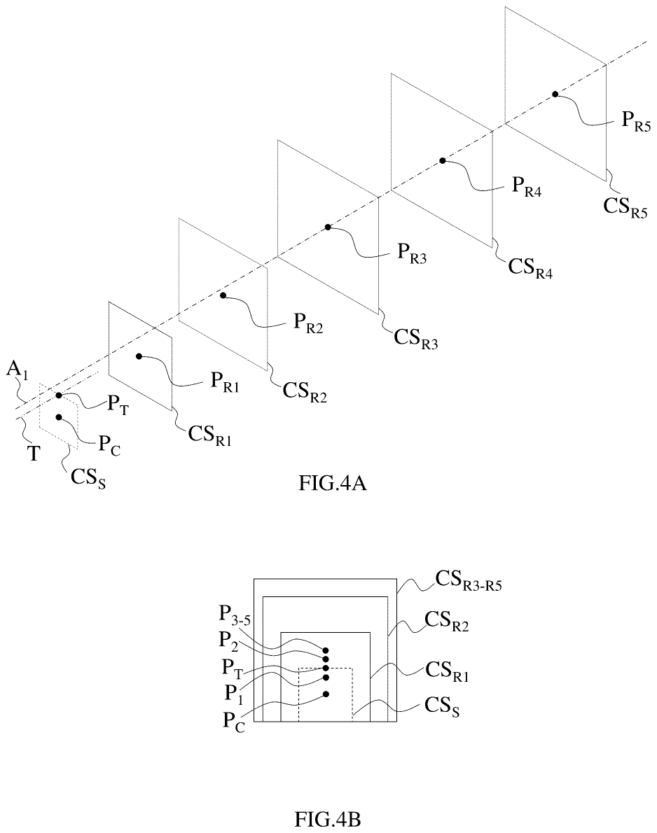

FIGS. 4A and 4B illustrate the first axis A₁ in detail. FIG. 4A shows the front, right side, and top views illustrating six cross-sections (standard cross-section CS_{S} and reference cross-sections CS_{R} (CS_{R1} to CS_{R5})) of the ophthalmic surgical instrument 1. FIG. 4B shows a set of front views of each cross-section shown in FIG. 4A. The cross-sections are parallel to each other, and their areas are as follows: CS_{S}<CS_{R1}<CS_{R2}<CS_{R3}=CS_{R4}=CS_{R5}. In FIGS. 4A and 4B, the center points P_{R3} to P_{R5} of the reference cross-sections CS_{R3} to CS_{R5} overlap most when viewed in a front view, and the first axis A₁ is an axis passing through the center points P_{R3} to P_{R5}.

FIGS. 4A and 4B illustrate five reference cross-sections (CS_{R1} to CS_{R5}) as an example, but are not limited thereto. In one embodiment, the plurality of reference cross-sections CS_{R} are selected from a region of the shaft having a constant cross-sectional size. If no such region of the shaft having the constant cross-sectional size exists, the first axis A₁ may be defined as an axis passing through the center point of a cross-section near the proximal end of the shaft and parallel to the horizontal tangent T.

### First Angle α₁, Second Angle α₂, and Tip Length L

FIG. 5 is a schematic enlarged right side view of the incising portion 20. In one embodiment, the incising portion 20 may include a first cutting surface 231 provided between the tip 21 and the bottom surface 23, and a second cutting surface 221 provided between the tip 21 and the top surface 22. In one embodiment, when viewed in a side view, the first angle α₁ defined between the first cutting surface 231 and the second cutting surface 221 is between 20° and 50° (e.g., 20°, 25°, 30°, 35°, 40°, 45°, and 50°, or a range between any two values selected from these values). In one embodiment, when viewed in a side view, the second angle α₂ defined between the second axis A₂ bisecting the first angle α₁ and the horizontal tangent T is between 60° and 100° (e.g., 60°, 65°, 70°, 75°, 80°, 85°, 90°, 95°, and 100°, or a range between any two values selected from these values) on the side of the top surface 22.

The tip length L (length of a perpendicular line) from the tip 21 of the incising portion 20 to the horizontal tangent T may be 0.40 μm to 0.50 μm (e.g., 0.40 μm, 0.41 μm, 0.42 μm, 0.43 μm, 0.44 μm, 0.45 μm, 0.46 μm, 0.47 μm, 0.48 μm, 0.49 μm, and 0.50 μm, or a range between any two values selected from these values). The tip length L is preferably 0.43 μm to 0.47 μm, and more preferably 0.44 μm to 0.46 μm.

In one embodiment, the point of intersection P_{T} is located on the side of the shaft rear surface 13 relative to the first axis A₁ when viewed in a top view. In another embodiment, the first axis A₁ is located between the center point P_{C} and the point of intersection P_{T} when viewed in a top view. The distance (length of a perpendicular line) from the point of intersection P_{T} to the first axis A₁ when viewed in a top view may be 0.001 μm to 0.020 μm, and may be 0.001 μm, 0.002 μm, 0.003 μm, 0.004 μm, 0.005 μm, 0.006 μm, 0.007 μm, 0.008 μm, 0.009 μm, 0.010 μm, 0.011 μm, 0.012 μm, 0.013 μm, 0.014 μm, 0.015 μm, 0.016 μm, 0.017 μm, 0.018 μm, 0.019 μm, and 0.020 μm, or a range between any two values selected from these values. The distance is preferably 0.005 μm to 0.015 μm, and more preferably 0.008 μm to 0.012 μm.

### Incision Range

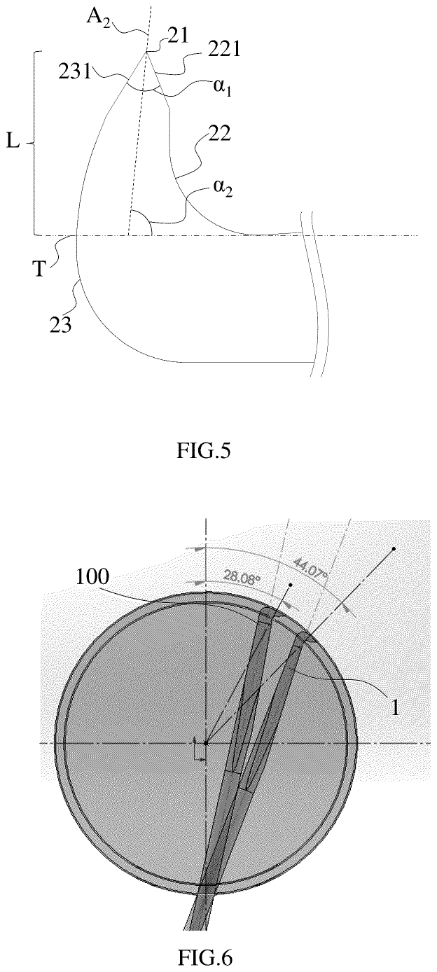

FIG. 6 is a schematic view showing incision ranges of the ophthalmic surgical instrument 1 according to this embodiment and a conventional ophthalmic surgical instrument 100. The conventional ophthalmic surgical instrument 100 has a structure in which the first axis A₁ passes through the center point P_{C} of the standard cross-section CS_{S}. It is noted that the tip length L of both the ophthalmic surgical instrument 1 according to this embodiment and the conventional ophthalmic surgical instrument 100 is 0.45 μm.

As shown in FIG. 6, the conventional ophthalmic surgical instrument 100 has an insertion angle of 28.08° per side (56.16° in total) along a virtual circle, whereas the ophthalmic surgical instrument 1 according to this embodiment has an insertion angle of 44.07° per side (88.14° in total) along the virtual circle. The incision range of the ophthalmic surgical instrument 1 according to this embodiment is 1.57 times wider than that of the conventional ophthalmic surgical instrument 100.

### Combination of Non-Glare Surface and Glare Surface

The top surface 22, bottom surface 23, shaft front surface 11, and/or shaft rear surface 12 may have surface roughness different from that of the incising side surface 24 and/or the shaft side surface 13. In one embodiment, the top surface 22, bottom surface 23, shaft front surface 11, and/or shaft rear surface 12 have a non-glare surface (matte surface), while the incising side surface 24 and/or the shaft side surface 13 have a glare surface (mirror-finished surface). In another embodiment, the top surface 22, bottom surface 23, shaft front surface 11, and/or shaft rear surface 12 have a glare surface (mirror-finished surface), while the incising side surface 24 and/or the shaft side surface 13 have a non-glare surface (matte surface). The non-glare surface can be formed by sandblasting, whereas the glare surface (mirror-finished surface) can be prevented from becoming non-glare during sandblasting by protecting each surface with a protective material such as masking tape.

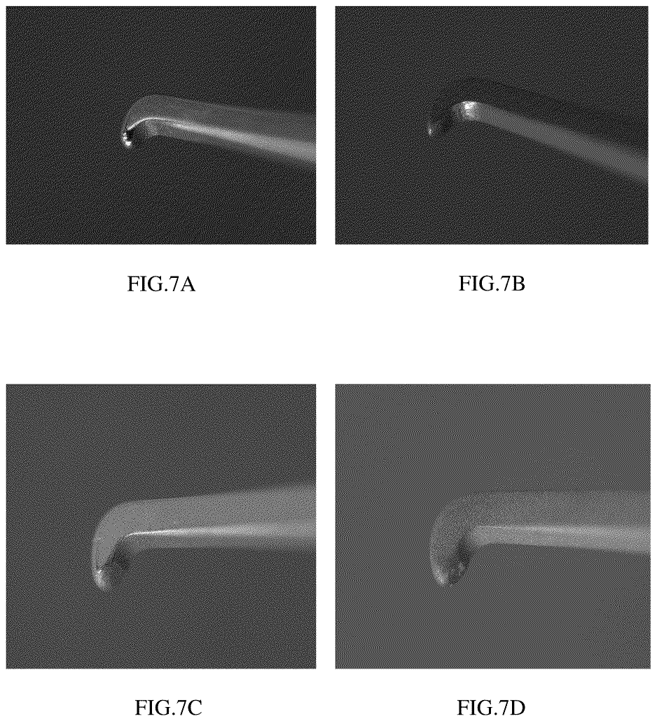

FIG. 7A is a photograph showing a distal end of shaft 1, whose entire surface is processed as a glare surface (mirror-finished surface). FIG. 7B is a photograph showing the distal end of shaft 1 where the top surface 22, bottom surface 23, shaft front surface 11, and shaft rear surface 12 are processed as non-glare surfaces (matte surfaces), and the incising side surfaces 24 and shaft side surfaces 13 are processed as glare surfaces (mirror-finished surfaces). FIG. 7C is a photograph showing the distal end of shaft 1, where the top surface 22, bottom surface 23, shaft front surface 11, and shaft rear surface 12 are processed as a glare surface (mirror-finished surface), and the incising side surfaces 24 and shaft side surfaces 13 are processed as a non-glare surface (matte surface). FIG. 7D is a photograph showing the distal end of shaft 1, whose entire surface is processed as a non-glare surface (matte surface). The ophthalmic surgical instrument 1 shown in FIGS. 7A and 7D has the same surface roughness on the top surface 22, bottom surface 23, shaft front surface 11, and shaft rear surface 12 as on the incising side surfaces 24 and shaft side surfaces 13. Consequently, there was no contrast difference between the side surfaces and the other surfaces, making it difficult to recognize the orientation of the ophthalmic surgical instrument 1 during surgery. In contrast, the ophthalmic surgical instrument 1 shown in FIGS. 7B and 7C has a different surface roughness on the top surface 22, bottom surface 23, shaft front surface 11, and shaft rear surface 12 compared to the incising side surfaces 24 and shaft side surfaces 13. This difference in roughness created a contrast between the side surfaces and the other surfaces, allowing the orientation of the ophthalmic surgical instrument 1 to be easily recognized during surgery.
1: Ophthalmic surgical instrument
2: Handle
10: Shaft
10A: Distal region
10B: Proximal region
11: Shaft front surface
12: Shaft rear surface
13: Shaft side surface
20: Cleaving portion
21: Tip
22: Top surface
23: Bottom surface
24: Cleaving side surface
100: Conventional ophthalmic surgical instrument
231: First cutting surface
221: Second cutting surface
A₁: First axis
A₂: Second axis
CS_{R}: Reference cross-section
CS_{S}: Standard cross-section
H: Horizontal axis
P: Center point
P_{C}: Center point of standard cross-section
P_{R}: Center point of reference cross-section
P_{T}: Point of intersection
T: Horizontal tangent
α₁: First angle
α₂: Second angle

What is claimed is:

1. An ophthalmic surgical instrument for incising fibrous tissue in the eye, the ophthalmic surgical instrument, comprising:
a shaft having a longitudinal axis; and
an incising portion projecting in a direction away from a longitudinal direction of the shaft,
wherein the shaft comprises:
a shaft front surface located on a side of a projection direction of the incising portion; and
a shaft rear surface located on the opposite side from the projection direction,
wherein the incising portion comprises:
a tip having a minimum thickness to which a thickness of the incising portion in a longitudinal direction of the shaft gradually decreases toward the projection direction;
a top surface being a connecting surface between the tip and the shaft front surface; and
a bottom surface being a connecting surface between the tip and the shaft rear surface, and
wherein the shaft having, in a side view, a standard cross-section being perpendicular to a horizontal tangent that first becomes parallel to a horizontal axis extending in the longitudinal direction of the shaft among tangents to a surface or a point transitioning from the top surface to the shaft front surface and passing through a point of intersection between the horizontal tangent and the top surface, and
a center point of the standard cross-section is positioned rearward, toward the shaft rear surface, relative to a first axis passing through most number of center points in a front view among center points of a plurality of reference cross-sections of the shaft parallel to the standard cross-section.

2. The ophthalmic surgical instrument according to claim 1, the shaft comprises a region having a constant thickness on the proximal side,
wherein the thickness of the shaft is smallest at the point of intersection and increases proximally from the point of intersection up to the region of constant thickness.

3. The ophthalmic surgical instrument according to claim 1, the incising portion further comprises:
a first cutting surface provided between the tip and the bottom surface; and
a second cutting surface provided between the tip and the top surface,
wherein, in a side view, a second angle defined between the first axis and a second axis bisecting a first angle defined between the first cutting surface and the second cutting surface is between 60 and 100 degrees on the side of the top surface.

4. An ophthalmic surgical instrument for incising fibrous tissue in the eye, the ophthalmic surgical instrument, comprising:
a shaft having a longitudinal axis; and
an incising portion projecting in a direction away from a longitudinal direction of the shaft,
wherein the shaft comprises:
a shaft front surface located on a side of a projection direction of the incising portion;
a shaft rear surface located on the opposite side from the projection direction; and
shaft side surfaces, each shaft side surface being a connecting surface between the shaft front surface and the shaft rear surface,
wherein the incising portion comprises:
a tip having a minimum thickness to which a thickness of the incising portion in a longitudinal direction of the shaft gradually decreases toward the projection direction;
a top surface being a connecting surface between the tip and the shaft front surface; and
a bottom surface being a connecting surface between the tip and the shaft rear surface;
incising side surfaces, each incising side surface being a connecting surface between the top surface and the bottom surface,
wherein the shaft having, in a side view, a standard cross-section being perpendicular to a horizontal tangent that first becomes parallel to a horizontal axis extending in the longitudinal direction of the shaft among tangents to a surface or a point transitioning from the top surface to the shaft front surface and passing through a point of intersection between the horizontal tangent and the top surface, and
a center point of the standard cross-section is positioned rearward, toward the shaft rear surface, relative to a first axis passing through most number of center points in a front view among center points of a plurality of reference cross-sections of the shaft parallel to the standard cross-section, and
wherein the top surface, bottom surface, shaft front surface and/or shaft rear surface comprise a surface roughness different from that of the incising side surfaces and/or shaft side surfaces.
